(19) United States
(12) Patent Application Publication
(10) Pub. No.: US 2021/0077797 A1
(43) Pub. Date: Mar. 18, 2021

(54) TRACK ENDOCERVICAL LOWER UTERINE MEDICINE INSERTER

(71) Applicant: Rufus Thrower, JR., Little Rock, AR (US)

(72) Inventor: Rufus Thrower, JR., Little Rock, AR (US)

(21) Appl. No.: 16/571,444

(22) Filed: Sep. 16, 2019

(51) Int. Cl. A61M 31/00

(52) U.S. Cl. CPC A61M 31/007; A61B 5/4343; A61M 2210/1433; A61M 2210/1475

(57) ABSTRACT

An illustrated view of an exemplary endocervical labor induction medicine injection apparatus for administering medication prior to or in the early stages of childbirth. The endocervical labor induction medicine injection apparatus is a medication apparatus that provides for controlled insertion of tablet style medication in an endocervical or lower uterine body area. The endocervical labor induction medicine injection apparatus provides increased functionality over the current manual placement methods ensuring maximum benefit and reduction of unwanted side effects. The endocervical labor induction medicine injection apparatus has a guide tube and an ejector tube. The ejector tube has an ejector head with a receiving cavity at a distal end of the ejector tube for receiving and dispensing the medication. A wire couples the receiving cavity at the distal end to a handle at a proximal end of the ejector tube. The guide tube is inserted into the cervical cavity of a patient. After determining proper positioning and loading of the medication into the receiving cavity, the distal end of the ejector tube is slid through the guide tube. The handle is then manipulated to release the medication at the desired location.

### FIELD OF THE INVENTION

This invention relates to endocervical/lower uterine body area medication. More particularly, it relates to devices to placement of medical tablets in the endocervical/lower uterine body area.

### BACKGROUND

Labor induction is the process or treatment that stimulates childbirth and delivery. Inducing labor can be accomplished with pharmaceutical or non-pharmaceutical methods. In Western countries, it is estimated that one-quarter of pregnant women have their labor medically induced with drug treatment. Inductions are most often performed either with prostaglandin drug treatment alone, or with a combination of prostaglandin and intravenous oxytocin treatment.

Methods of inducing labor include both pharmacological medication and mechanical or physical approaches.

Mechanical and physical approaches can include artificial rupture of membranes or membrane sweeping. The use of intrauterine catheters are also indicated. These work by compressing the cervix mechanically to generate release on prostaglandins in local tissues. There is no direct effect on the uterus.

Pharmacological methods include dinoprostone (prostaglandin E2), misoprostol (a prostaglandin E1 analogue), and intravenous oxytocin.
Intravaginal, endocervical or extra-amniotic administration of prostaglandin, such as dinoprostone or misoprostol. Prostaglandin E2 is the most studied compound and with most evidence behind it. A range of different dosage forms are available with a variety of routes possible.

A common procedure in the obstetric/gynecology field is the placement of tablet style medication into the endocervical/lower uterine cavity. Such medicines are used for induction of labor, control of excessive uterine bleeding and other medical needs. Whatever the reason, such tablets are typically inserted manually by doctor or other medical personnel. As such, exact placement of such medicine is typically a hit or miss process. Should the tablet not be exactly placed, not only is it not effective and not productive of the desired effect, but it can also affect other areas of the body in an unintended manner thus producing unwanted side effects.

In light of the above noted problems, there exists a need for a means by which medicinal tablets can be quickly an accurately placed in the endocervical/lower uterine body cavity without difficulties and/or risks noted above.

### BRIEF DESCRIPTION OF THE DRAWINGS

FIG. 1 an illustrated view of an exemplary endocervical labor induction medicine injection apparatus.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

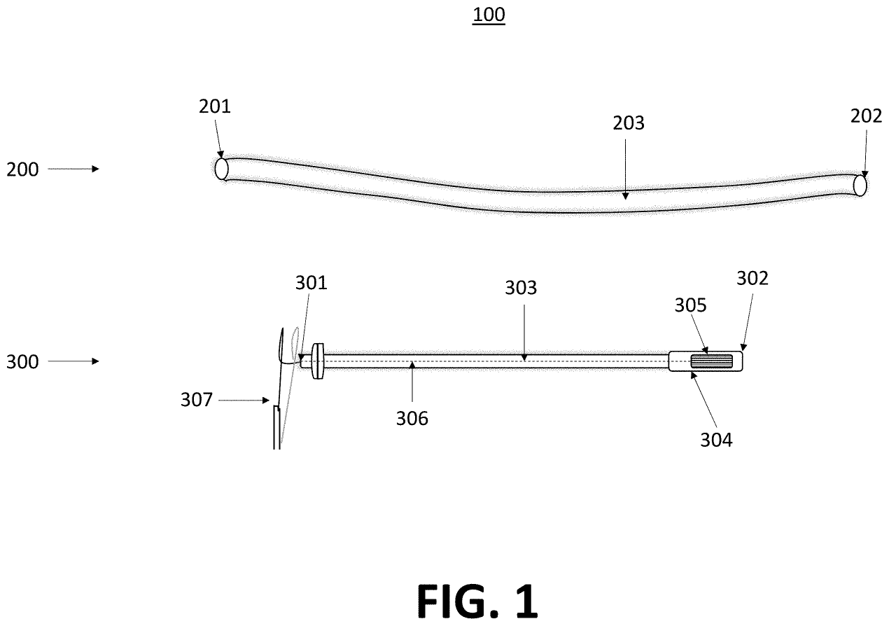

Referring to FIG. 1, an illustrated view of an exemplary endocervical labor induction medicine injection apparatus 100 for administering medication prior to or in the early stages of childbirth. The endocervical labor induction medicine injection apparatus 100 is a medication apparatus that provides for controlled insertion of tablet style medication in an endocervical or lower uterine body area. The endocervical labor induction medicine injection apparatus 100 provides increased functionality over the current manual placement methods ensuring maximum benefit and reduction of unwanted side effects.

The endocervical labor induction medicine injection apparatus 100 has a guide tube 200 and an ejector tube 300.

The guide tube 200 preferably has a diameter of ten (10) millimeters (mm), however other diameters are hereby contemplated, including, but not limited to, eight (8) mm, twelve (12) mm, etc. The guide tube 200 preferably has a length of twenty-eight (28) centimeters (cm), however other lengths are hereby contemplated, including, but not limited to, twenty-six (26) cm, thirty (30) cm, etc. The guide tube 200 is preferably made of a plastic material, however other materials are hereby contemplated, including, but not limited to, high-density poly-ethylene (HDPE), low-density poly-ethylene (LDPE), etc. The material must be medical grade. The guide tube 200 is preferably malleable and/or flexible.

The guide tube 200 has a first end 201, a second end 202 and an interior portion 203. The first end 201 of the guide tube 200 and the second end 202 of the guide tube 200 are open, thereby providing an ingress/egress access to the interior portion 203 of the guide tube 200. The guide tube 200 is useful to be used to place into a patient and create a pathway to a desired location to place the medication.

The ejector tube 300 is useful for being configured to hold a tablet style medication and deliver the medication to a desire location in a patient. The ejector tube 300 is preferably made of a plastic material, however other materials are hereby contemplated, including, but not limited to, high-density poly-ethylene (HDPE), low-density poly-ethylene (LDPE), etc. The material must be medical grade. The ejector tube 300 is preferably malleable and/or flexible. The ejector tube 300 preferably has a length of thirty (30) cm, however other lengths are hereby contemplated, including, but not limited to, twenty-eight (28) cm, thirty-two (32) cm, etc. The ejector tube 300 must be at least two (2) cm longer than the guide tube 200.

The ejector tube 300 has a proximal end 301, a distal end 302 and a middle portion 303. The distal end 302 has an ejector head 304. The ejector head 304 is configured to hold and release a tablet like medication upon demand by physician or medical personnel. The ejector head 304 has a receiving cavity 305, the receiving cavity 305 being for containing the tablet-like medication.

The ejector tube 304 is preferably thirty (30) cm in length, however other lengths are hereby contemplated, including, but not limited to, high-density poly-ethylene (HDPE), low-density poly-ethylene (LDPE), etc. The ejector tube 304 is preferably seven (7) cm in circumference, however other circumferences are hereby contemplated, including, but not limited to, six (6) cm, eight (8) cm.

The ejector head 304 is preferably three (3) cm in length, however other lengths are hereby contemplated, including, but not limited to, two (2) cm, four (4) cm, etc. The receiving cavity 305 is preferably one (1) cm in depth, however other depths are hereby contemplated, including, but not limited to, three-fourths (0.75) cm, one and one-fourth (1.25) cm, etc. The ejector head 304 preferably has a circumference of eight (8) cm, however other circumferences are hereby contemplated, including, but not limited to, seven (7) cm, nine (9) cm, etc. The circumference of the ejector head 304 must be at least the same circumference as the ejector tube 300. The circumference of the guide tube 200 must be larger than the circumference of the ejector tube 300 and the ejector head 304.

A wire 306 is configured to run through the middle portion 303 of the ejector tube 300. The wire 306 is coupled to the receiving cavity 305. The wire 306 extends past the proximal end 301 of the ejector tube forming a handle 307. The handle 307 provides for the manipulation of the wire 306. The manipulation of the wire 306 provides for the release of the tablet-like medication contained in the receiving cavity 305 when the medication can be released at the desired location.

To use the endocervical labor induction medicine injection apparatus 100, the second end 202 of the guide tube 200 is inserted into a cervical cavity of the patient and checked for proper positioning. Next, the receiving cavity 305 of the ejector head 304 of the ejector tube 300 is loaded with the proper medication. Once the medication is properly loaded inside of the receiving cavity 305, then the distal end 302 of the ejector tube 300 is placed through the first end 201 of the guide tube 200 until it exits through the second end 202 of the guide tube 200.

Once the receiving cavity 304 of the ejector tube 300 has been checked to ensure proper positioning for the tablet-like medication, the handle 307 is manipulated and the wire 306 is used to release the medication from the receiving cavity 305 of the ejector head 304 of the ejector tube 300. Once the medication has been properly placed the guide tube 200 and the ejector tube 300 may be removed from the cervical cavity of the patient.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An endocervical labor induction medicine injection apparatus for providing placement of tablet-like medication in the endocervical/lower uterine body area, the device comprising:
a guide tube, the guide tube being hollow;
a ejector tube, the ejector tube comprising:
a proximal end, a distal end and a middle portion;
an ejector head, the ejector head being coupled significantly near the distal end;
a receiving cavity, the receiving cavity being configured in the ejector head, wherein the receiving cavity being for holding the tablet like medication;
a wire, the wire coupled to the receiving cavity, the wire being extend past the proximal end of the ejector tube, wherein the wire forms a handle, and wherein the wire being configured to the middle portion of the ejector tube.

2. The device of claim 1, wherein the ejector tube having a circumference of seven (7) centimeters (cm).

3. The device of claim 1, wherein the ejector head having a circumference of eight (8) cm.

4. The device of claim 1, wherein the receiving cavity having a depth of one (1) cm.

5. The device of claim 1, wherein the ejector head having a length of three (3) cm.

6. The device of claim 1, wherein the ejector tube having a length of thirty (30) cm.

7. The device of claim 1, wherein the guide tube having a circumference being ten (10) cm.

8. The device of claim 1, wherein the guide tube having a length of twenty-eight (28) cm.

9. The device of claim 1, wherein the ejector tube having a greater length than the guide tube.

10. The device of claim 1, wherein the ejector head having at least the same circumference as the ejector tube.

11. The device of claim 1, wherein the guide tube being made of a plastic material.

12. The device of claim 11, wherein the plastic material being medical grade plastic.

13. The device of claim 1, wherein the ejector tube being made of a plastic material.

14. The device of claim 13, wherein the plastic material being medical grade plastic.
